(19) United States
(12) Patent Application Publication
(10) Pub. No.: US 2009/0001622 A1
(43) Pub. Date: Jan. 1, 2009

(54) METHODS FOR MOLDING INTERBODY DEVICES IN SITU

(76) Inventors: Remi Nader, Greenwood, MS (US); Jimmy D. MILLER, Greenwood, MS (US)

Correspondence Address: BROWDY AND NEIMARK, P.L.L.C.;624 NINTH STREET, NW, SUITE 300, WASHINGTON, DC, 20001-5303, US

(21) Appl. No.: 11/769,076

(22) Filed: Jun. 27, 2007

(51) Int. Cl. B29B 9/00; A61B 17/58

(52) U.S. Cl. 264/5; 606/102

(57) ABSTRACT

A method is provided for producing and inserting a cervical interbody mold device (CIMD). The CIMD produces an interbody device that is formed in situ and that possesses suitable strength and biocompatibility so as to provide sufficient vertebral support while providing optimal ease of use and insertion for the surgeon.

### FIELD OF THE INVENTION

One of the most costly health problems in society involves back pain and pathology of the spine. These problems can affect individuals of all ages and can result in great suffering to victims. Back pain can be caused by several factors, such as congenital deformities, traumatic injuries, degenerative changes to the spine, and the like. Such changes can cause painful excessive motion, or collapse of a motion segment resulting in the contraction of the spinal canal and compressing the neural structures, causing debilitating pain, paralysis, or both, which in turn can result in nerve root compression or spinal stenosis.

Intervertebral discs, disposed between endplates of adjacent vertebrae, both stabilize the spine and cushion vertebral bodies. However, diseased, degenerated, displaced, or otherwise damaged discs (e.g., herniated or ruptured discs) manifest numerous undesirable symptoms, including nerve damage, pain, spinal instability, numbness and decreased mobility.

Often surgical intervention, in the nature of a discectomy, is necessary to correct the aforementioned symptoms. In this procedure, the involved vertebrae are exposed and the intervertebral disc is removed, thus removing the offending tissue or providing access for removing bone osteophytes. A second procedure, termed a spinal fusion, may then be required to fix the vertebrae together to prevent movement and maintain a space originally coupled by the intervertebral disc.

During a spinal fusion following a discectomy, a prosthetic implant or spinal implant is inserted into the intervertebral space. This implant can be a bone graft removed from another portion of the patient's body, termed an autograph. The use of an autograph has the important advantage of avoiding rejection of the implant, but it also has several shortcomings. There is always a risk in opening a second surgical site to obtain the implant, which can lead to infection or pain for the patient, and the site of the implant is weakened by the removal of bony material. The bone implant may not be perfectly shaped and placed, leading to slippage or absorption of the implant or failure of the implant to fuse with the vertebrae.

Other sources for a graft source of the implant are bone removed from cadavers, termed allograft, or from other species, termed xenograft. In these cases, while there is the benefit of not requiring a second surgical site as a possible source of infection or pain, there is increased likelihood of graft rejection and a risk of transmitting communicable diseases.

An alternative approach is to use a manufactured implant made of a synthetic material that is biologically compatible with the body and the vertebrae. Several compositions and geometries of such implants have been used, ranging from simple blocks of material to carefully shaped implants, with varying success. Optimally, these devices provide temporary support while permitting the ingrowth of new bone.

Prosthetic implants of this nature are generally solid implants or implants designed to encourage bone ingrowth. It has been found that devices that permit bone to grow across or through the implant achieve a more rapid and stable arthrodesis than the solid implants. These implants are generally filled with autologous bone prior to insertion into the intervertebral disc space. These implants typically include apertures which communicate with opening in the implant, thereby providing a path for tissue growth between the vertebral end plate and the bone or bone substitute within the implant.

Suitable materials include acrylic poly methyl methacrylate (PMMA), titanium, carbon fiber, hydroxyl apatitites, and biopolymers such as xenografts and resorbable cages (Clinical Neurosurgery 52:197). For example, in U.S. Pat. No. 6,958,078 to Goel et al.; U.S. Pat. No. 6,758,863 to Estes et al.; U.S. Pat. No. 6,786,930 or Biscup and U.S. Published Application Nos. 2005/0177245 to Leatherbury et al. and 2006/0025861 to McKay, all disclose molded implants comprised of polymers or other suitable material.

PMMA has been widely used as an interbody device for treating degenerative disc disease of the cervical spine, as reported by Grote, Acta Neurichir 16:218-240, 1967. However, when PMMA in liquid form was poured into the disk space and allowed to harden, to form a stand alone device, the device was demonstrated not to promote the most optimal fusion based on radiographic result as compared with discectomy alone (Clinical Neurosurgery 52:197). More recent studies, however, have demonstrated better fusion results employing different techniques with PMMA Clinical Neurosurgery 52:197); Zentralbl Neurochir. 62(2): 33-36, 2001). One study completed in 2005 revealed a 90.5% fusion rate at six months and 100% at twelve months with interbody devices composed of prefabricated PMMA rings filled with autologous cancellous bone (Clinical Neurosurgery 52:197).

Many newer intervertebral cages permit some manipulation of the cage itself, so as to provide a better fit within a patient's intervertebral space. For example, U.S. Pat. No. 6,471,725, to Ralph et al., discloses the use of a variety of sized spacers in sequence to widen the intervertebral space during an implantation procedure. The above-cited patent to Biscup provides for customized implants manufactured by transferring information regarding the proposed site of implantation to the implant molding machine. However, these prefabricated spacers are often exceedingly and unnecessarily expensive. Thus, there remains a need for surgical procedures that permit rapid manufacture and configuration of an interbody device during the discectomy itself, rather than prior to the operation, wherein the surgeon is able to construct the appropriate implant precisely and efficiently despite the time, equipment and resource constraints imposed by the surgical environment.

### SUMMARY OF THE INVENTION

The method as described herein is designed to overcome the aforesaid deficiencies in the prior art.

The method herein provides a method for producing and inserting a cervical interbody mold device (CIMD) that eliminates the need for an iliac crest incision for bone graft harvesting, thus reducing surgical risk and operative time.

The method as described herein provides a CIMD of suitable strength and biocompatibility so as to provide sufficient vertebral support while providing optimal ease of use and insertion for the surgeon.

The method described herein provides an implantable device of suitable strength and biocompatibility so as to provide orthopedic support while providing optimal ease of use and insertion for the surgeon, including but not limited to spine implants, joint implants, and other orthopedic implants.

The CIMD implant materials described herein are those that have a proven safety and efficacy record for use in spinal and other weight-bearing prostheses. Among these materials is PMMA. The materials used herein are preferably economical materials such as PMMA, rather than more expensive spacers constructed of polyetheretherketones(PEEK), bone autograft, or various other prefabricated plastics. PMMA is preferred because of its safety, efficacy and cost

The present IMD is made wholly or primarily of synthetic material that obviates any risk or expense stemming from inadequate processing or acquisition of donor bone grafts.

When produced as described herein, the IMD permits placement of the bone graft or demineralized bone matrix in the center of the disc space. The present method does not, in contrast to some prior methods, entail pouring PMMA or other material directly into the disc space. This also eliminates any risk of leakage of PMMA across the posterial longitudinal ligament, which has the potential to cause damage to the proximal nerve roots or spinal cord and possibly further spinal cord stenosis.

The present IMD device can be readily sterilized between uses and used a plurality of times, wherein only the PMMA component need be replaced for subsequent procedures.

Additionally, the IMD does not require predetermination of interbody spacer size, unlike previous devices that use precut pieces of bone allograft or prefashioned PEEK spacers. The surgeon or technician can create several interbody spacers with the same portion of PMMA. The interbody mold device herein comprises a stand-alone mold that provides interbody spacers in cervical spine discectomy and fusion procedures and similar surgeries, as well as efficient methods for creating these spacers during the surgery, and then inserting these spacers into a patient's vertebral disc. The herein claimed device can be manipulated into various configurations, customized and tailored to the specific disc space dimensions of individual patients.

The IMD is composed of two separated parts. When these parts are combined together, they form a stand-alone mold. A material for producing an interbody spacer, such as PMMA, is introduced into the mold and allowed to harden. Once the material is hardened, the mold is separated for release.

Since PMMA has long been used as an implant in various parts of the body, including long bones, spine, and weight bearing parts, for over 30 years, and has proven to be a safe material with inert properties, it is particularly well suited for making cage implants.

Using PMMA avoids problems with the safety of use of donor bond grafts with respect to inadequate processing or acquisition. Since PMMA hardens very rapidly, within minutes, there is no significant delay in producing an implant ready for placement in situ during the surgical procedure.

Using the CIMD described herein is greatly advantageous over pouring PMMA directly into the disc space, as the latter technique does not provide the ability to place bone graft or demineralized bone matrix in the center of the disc space. Pouring PMMA directly into the disc space may potentially damage nerve root or spinal cord because of heat via exothermic reaction, physical compression, or chemical damage (Mt. Sinai J. Med 61(3); 246-247, 1994; J Neurosurg 98 (Suppl): 21-30, 2003).

In the instant process, a small portion of polymethylmethacrylate is used to create an interbody device. This process significantly reduces the cost of the fusion device to merely a fraction of the cost of prefabricated spacers.

### BRIEF DESCRIPTION OF THE DRAWINGS

FIGS. 1A, B and C illustrate the CIMD.

FIGS. 2A, B and C illustrate the CIMD for two different interbody spacer heights.

FIG. 3 represents the CIMD for four different sizes of interbody spacers. FIG. 3A is a top view, FIG. 3B is a side view, and FIG. 3C is an end view.

FIG. 4, Steps 1, 2 and 3, illustrates the process of making a spacer and placing it into the disc space.

FIG. 5 illustrates cross sections of CIMD devices with modified spacer angulation (FIG. 5A) and a spacer with corrugated edges (FIG. 5B).

### DETAILED DESCRIPTION OF THE INVENTION

For purposes of the present application, CIMD can be used interchangeably with IMD. It is clear that the method and device are not limited to cervical procedures, but can be used for all relevant orthopedic and neurosurgical procedures.

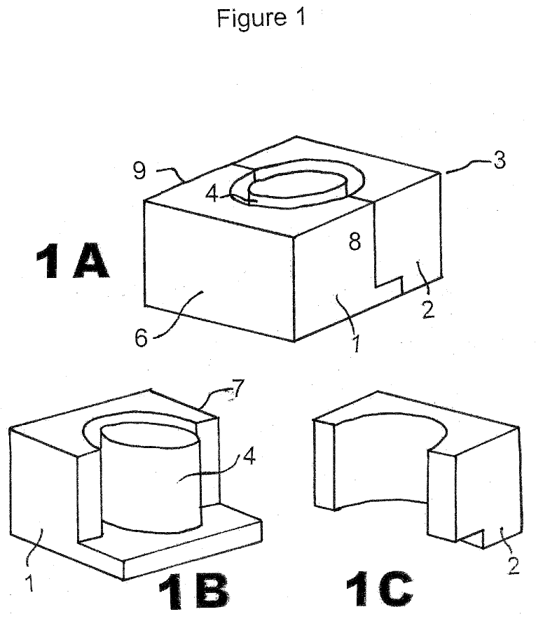

The CIMD 3 is composed of two parts 1, 2 which, when combined together as shown in FIG. 1A, form a stand-alone mold which can be separated for release after hardening of the interbody spacer created in situ after separation from its inner cylinder mold 4. Generally, the CIMD has a pair of flat side faces and inner faces formed of half cavities.

FIG. 1A shows the CIMD 3 closed (parts 1 and 2 combined together by apposition). FIGS. 1B and 1C demonstrate the CIMD 3 open as part 1 (FIG. 1B) and part 2 (FIG. 1C) are separated. A form 10 is in the center of the closed CIMD so that the spacer formed is open on the inside.

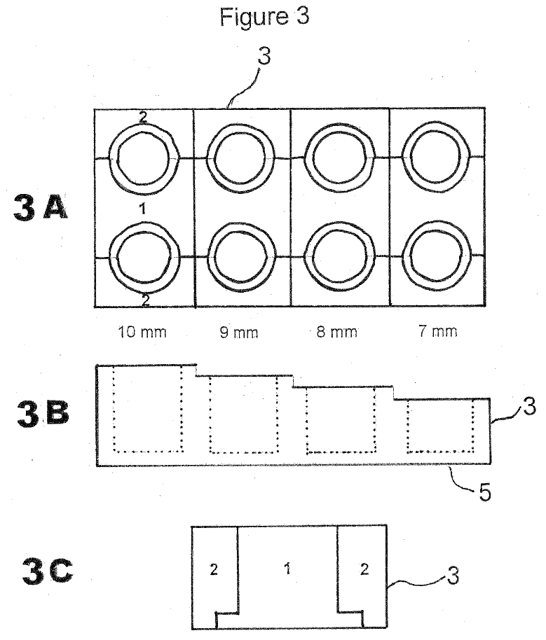

Typically, the CIMD 3 when assembled includes a top wall 4, a bottom wall 5, shown in FIG. 3B, a first end wall 6, a second end wall 7, a front side wall 8, and a back side wall 9.

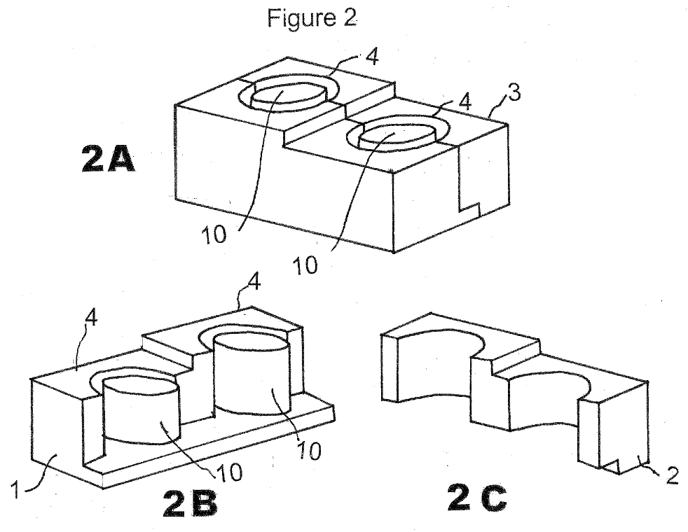

FIGS. 2A, 2B and 2C illustrate the CIMD for two different interbody spacer heights. FIG. 2A shows the device 3 closed. FIGS. 2B and 2C demonstrate the device 3 open as part 1 and part 2 are separated. This figure represents the device 3 with two spacer sizes of different heights with similar inner and outer diameters (same interbody spacer thickness).

FIG. 3 shows the CIMD 3 for four different sizes of interbody spacers 4, in this case ranging from 6 to 10 mm. The mold has been duplicated to produce a total of eight spacers. This permits the surgeon to create two spacers of the same size simultaneously. FIG. 3A is a top view, FIG. 3B is a side view, and FIG. 3C is an end view.

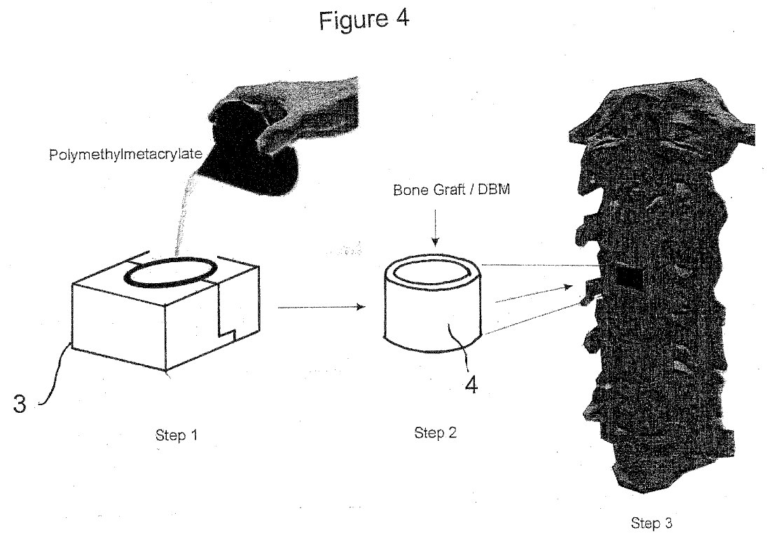

FIG. 4 illustrates the process for making a spacer and placing it into the disc space. In step 1, polymethylmethacrylate is poured into the device 3. Once the PMMA has hardened, in step 2 the CIMD 3 is opened and the completed spacer 4 is removed. Optionally, the surgeon places bone graft or demineralized bone matrix inside the spacer prior to in situ placement. In step 3, the spacer 4 is positioned in the spine 5 intraoperatively at the disc space after discectomy is completed. While the spacer shown here is generally of a cylindrical shape, any shape that fits into the space as measured within an individual patient can be used.

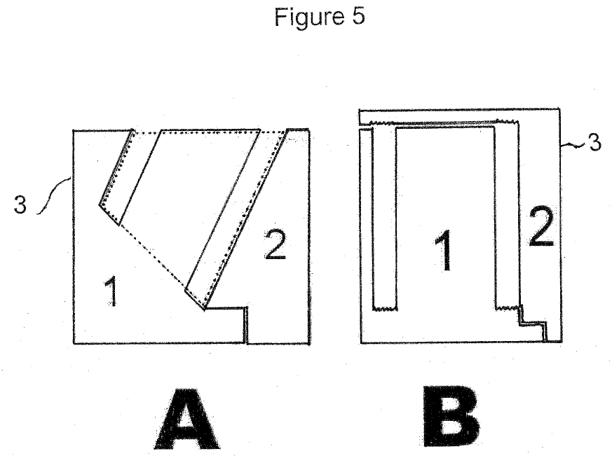

FIG. 5 demonstrates cross sections of CIMD devices 3 with modified spacer angulations (FIG. 5A) and a spacer with corrugated edges (FIG. 5B). These represent variations of the CIMD, but are not limiting as to the configurations that can be used. Parts 1 and 2 are separated as shown in FIGS. 1 and 2. The hatched marks represent location and shape of the spacer created.

The CIMD provides a method for producing intervertebral bodies for disc replacement. An anterior cervical discectomy is performed using standard techniques, such as the Cloward technique (J. Neurosurg 15:602-617, 1958). Once the disc material is resected using a combination of a high speed drill and kerrison rongeurs, a rectangular space is created at the disc space by squaring off the uncovertebral joints laterally, which may be done with the high speed drill. The end plates are also prepared by using the high speed drill to remove any remaining disc or cartilage, hence creating two smooth parallel straight surfaces, in a similar fashion to the described standard operation of discectomy and fusion. The posterior longitudinal ligament may optionally be removed, at the surgeon's preference.

The disc space is then measured with calipers or interbody spacers, and the closest fitting mold on the CIMD device is then selected. At this time, the PMMA powder and initiator solution are mixed for preparing for pouring into the mold.

The PMMA solution which may contain optional fillers, which is relatively viscous, is poured into the selected CIMD space. The pouring is done up to the edge of the CIMD surface, and any excess is promptly wiped off the surface. Once the PMMA hardens, the mold is opened and the spacer is removed from the mold.

The spacer so created was then packed densely with selected osteoinductive material, including bone graft, demineralized bone matrix, or bone morphogenic protein. Other bone growth factors and additives can be added at the surgeon's discretion. The spacer is then positioned in place in the appropriately created disc space. The fusion may then be enhanced by anterior instrumentation with plate and screws, at the surgeon's discretion.

While PMMA is currently the material of choice for making spacers as claimed herein, other materials that are physiologically compatible and have the requisite strength can also be used. Among these other materials are PEEK and other plastics and metals. Additionally, PMMA can be the filler in harder materials. Among the other materials that can be used to produce the implants are polymers filled with titanium, carbon fiber, hydroxyl apatites, and biopolymers such as xeongrafts and resorbable polymers, as long as these materials can be molded quickly during a surgical procedure.

The technique described herein can be used to create interbody mold devices for thoracic and lumbar spine procedures, such as thoracic diskectomy, lumbar interbody fusion procedures, both anterior and posterior interbody fusion and similar implants, such as spine implants, joint implants, and other orthopedic implants.

It is to be understood that the phraseology or terminology employed herein is for the purpose of description and not of limitation. The means and materials for carrying out various disclosed functions may take a variety of alternative forms without departing from the invention.

Thus, the expressions “means to . . . ” and “means for . . . ” as may be found in the specification above and/or in the claims below, followed by a functional statement, are intended to define and cover whatever structural, physical, chemical, or electrical element or structures which may now or in the future exist for carrying out the recited function, whether or nor precisely equivalent to the embodiment or embodiments disclosed in the specification above. It is intended that such expressions be given their broadest interpretation.

What is claimed is:

1. A method for producing a custom interbody device using an interbody mold device during surgery (intraoperatively), comprising:
a. measuring the interbody space and choosing an interbody mold device that fits the interbody space;
b. pouring a polymerizable fluid into the interbody mold device;
c. allowing the polymerizable fluid to harden;
d. opening the interbody mold device; and
e. removing the custom interbody device from the mold.

2. The method according to claim 1 wherein the interbody device is packed with osteoinductive material prior to being positioned in place.

3. The method according to claim 2 wherein the osteoinductive material is selected from the group consisting of bone graft, demineralized bone matrix, and bone morphogenic protein.

4. The method according to claim 1 wherein the polymerizable fluid is a polymethylmethacrylate.

5. The method according to claim 1 wherein the interbody mold device is a cervical interbody mold device.

6. The method according to claim 1 wherein the interbody mold device is also for thoracic and lumbar procedures.

7. The method according to claim 1 wherein the polymerizable fluid is PEEK.
